(19) United States
(12) Patent Application Publication
(10) Pub. No.: US 2010/0249452 A1
(43) Pub. Date: Sep. 30, 2010

(54) METHOD FOR THE OXIDATION OF CYCLOALIPHATIC ALCOHOLS, CYCLOALIPHATIC KETONES, OR MIXTURES THEREOF WITH AQUEOUS NITRIC ACID AND TREATMENT OF THE DICARBOXYLIC ACIDS

(75) Inventors: Thomas Jaworek, Kallstadt (DE); Christian Miller, Deidesheim (DE); Manfred Stroezel, Ilvesheim (DE); Günther Achhammer, Mannheim (DE); Thomas Papkalla, Mannheim (DE); Wilfried Berning, Worms (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/738,861

(22) PCT Filed: Oct. 21, 2008
(86) PCT No.: PCT/EP08/64160

§ 371 Date: Apr. 20, 2010

(30) Foreign Application Priority Data
Oct. 22, 2007 (DE) 10 2007 050 713.7
Feb. 19, 2008 (DE) 10 2008 009 992.9

(51) Int. Cl. C07C 51/23

(52) U.S. Cl. 562/509

(57) ABSTRACT

A process for preparing dicarboxylic acids by oxidizing cycloaliphatic alcohols, cycloaliphatic ketones or mixtures thereof with nitric acid, by performing the reaction and separation of the components in a fractionating column or rectification column.

The present invention relates to a process for oxidizing cycloaliphatic alcohols, cycloaliphatic ketones or mixtures thereof with aqueous nitric acid and workup of the dicarboxylic acids by distillation.

Dicarboxylic acids can be synthesized by oxidizing cycloaliphatic alcohols, cycloaliphatic ketones or mixtures thereof in aqueous nitric acid with the aid of oxygenous gases.

US-A-3 754 024 discloses a semicontinuous process for preparing dicarboxylic acids by oxidizing cycloaliphatic alcohols, cycloaliphatic ketones or mixtures thereof in aqueous 40 to 70% nitric acid, in which the reaction is performed in an evaporator apparatus under boiling conditions at temperatures of from 70 to 80° C. at from 150 to 200 mbar. Connected to the evaporator apparatus is a column attachment in which the volatile components, nitric acid and steam, are separated by distillation. The evaporation rate is controlled by the supply of reactant solution.

DE-A-30 35 809 discloses a process for continuously preparing saturated, aliphatic polycarboxylic acids by oxidizing cycloaliphatic alcohols, cycloaliphatic ketones or mixtures thereof with nitric acid in the presence of an oxidation catalyst at elevated temperature and reduced pressure in a boiling reactor with attached rectification column and nitrogen oxide-containing entrainment gas.

The aforementioned processes left something to be desired.

It was therefore an object of the present invention to develop an improved process.

Accordingly, a novel and improved process has been found for preparing dicarboxylic acids by oxidizing cycloaliphatic alcohols, cycloaliphatic ketones or mixtures thereof with nitric acid, which comprises performing the reaction and separation of the components in a fractionating column or rectification column.

The process according to the invention can be performed as follows:

At a temperature of from 40 to 120° C., preferably from 60 to 90° C., more preferably from 70 to 80° C., and a pressure of from 1 to 2000 mbar, preferably from 50 to 300 mbar, more preferably from 100 to 200 mbar, cycloaliphatic alcohols, cycloaliphatic ketones or mixtures thereof can be reacted with nitric acid in the presence of a catalyst in a fractionating column, reaction column and/or rectification column.

The process according to the invention can be performed batchwise or continuously, preferably continuously.

Suitable fractionating columns, reaction columns and/or rectification columns are generally those columns with or without, preferably with, internals, which comprise from 1 to 150, preferably from 2 to 100, more preferably from 3 to 50 and especially 4 to 20 theoretical plates (trays). The fractionating columns, reaction columns and/or rectification columns comprise generally at least three (3), i.e. from 3 to 20, preferably from 3 to 10, more preferably from 3 to 6 and especially 3 segments which are provided with internals and which directly follow one another or are preferably provided with intermediate spaces. The individual segments may be internally divided and comprise different internals. The intermediate spaces generally have a length ratio relative to the segment below it of from 0.01:1 to 10:1, preferably from 0.1:1 to 5:1, more preferably from 0.2:1 to 1:1. In general, the fractionating columns, reaction columns and/or rectification columns comprise a bottom section and a top section which generally comprises no internals.

The distillation is a thermal separating process. In this process, the separation of a liquid mixture by partial evaporation of the mixture and subsequent condensation of the mixture vapor is achieved. The vapor above the boiling liquid mixture comprises more low-boiling components than the liquid mixture. When the vapor is drawn off and condensed, the distillate is richer in lower boilers than the liquid mixture used. The distillation process consists of evaporating and condensing. In the simplest case, the process consists of simple batchwise evaporation and the spatially separate condensation of the evaporated mixture vapor. The fundamental principle of distillative separation is the different vapor pressures of the mixture components. The substances with the higher vapor pressure preferably reside in the vapor phase. By virtue of simple distillation, an enrichment of the volatile component in the distillate is thus obtained. Simple distillation thus affords a concentration. In order to obtain a better separation, the distillation is performed repeatedly, and vapor and liquid are conducted in countercurrent. This process is also referred to as rectification or else fractional distillation. When a chemical reaction is simultaneously superimposed on the separation process, this is a reactive distillation, also known as reactive rectification.

The most important constituents of a rectification column is the rectification column or else column, known and also the column top and the column bottom. The column consists generally of a column jacket and column internals which ensure very intensive mass transfer and heat transfer. Any continuous column has at least one feed, a top draw and a bottom draw. The column section above the feed is referred to as the rectifying section, and that below the feed as the stripping section. Moreover, if required, further sidestreams can be withdrawn at different points between the top and bottom. The lowermost part of the column below the column internals is referred to as the column bottom, and the uppermost part above the internals as the column top. The most important additional apparatus required are an evaporator for heating or for generating the vapor phase and one condenser or else a plurality of condensers for cooling or condensing the vapors. In order to achieve the countercurrent flow of gas and liquid in the column, some of the vapors have to be recycled to the column top in condensed form.

It has been found to be particularly advantageous when from 1 to 5 and preferably from 2 to 4 theoretical plates are provided in the reaction zone above the bottom, from 1 to 5 and preferably from 2 to 4 theoretical plates in the middle zone, and from 1 to 10 and preferably from 2 to 7 theoretical plates in the upper part of the column below the top of the column above the feed stream.

Suitable internals are generally fillings of Raschig rings, Pall rings, Berl saddles (various manufacturers, for example Raschig), and also Hy-Pak and Intalox random packings (from Norton), Top-Pak and VSP random packings (from Vereinigte Füllkörper-Fabriken) or Hiflow Rings (from Reuscher), and any other kind of random packings. Structured packings such as SULZER Mellapak, CY, BX, DX or EX packing, Montz Pak in the A, B or C design, Koch Flexipak, Rombopak or any other kind of structured packings, and also separating trays, for example perforated trays, bubble-cap trays, valve trays, dual-flow trays, tunnel-cap trays in single-flow or multiflow design, preferably bubble-cap or tunnel-cap trays, more preferably tunnel-cap trays in the Thormann® tray design from Montz.

Suitable dicarboxylic acids are oxalic acid, malonic acid, succinic acid, glutaric acid, adipic acid, heptanedioic acid, octanedioic acid, nonanedioic acid, decanedioic acid, undecanedioic acid, dodecanedioic acid, preferably succinic acid, glutaric acid, adipic acid and dodecanedioic acid, more preferably adipic acid and dodecanedioic acid.

Suitable cycloaliphatic ketones are cyclobutanone, cyclopentanone, cyclohexanone, cyclooctanone, cyclononanone, cyclodecanone, cycloundecanone, cyclododecanone, preferably cyclobutanone, cyclopentanone, cyclohexanone and cyclododecanone, more preferably cyclohexanone and cyclododecanone.

Suitable cycloaliphatic alcohols are cyclobutanol, cyclopentanol, cyclohexanol, cyclooctanol, cyclononanol, cyclodecanol, cycloundecanol, cyclododecanol, preferably cyclobutanol, cyclopentanol, cyclohexanol and cyclododecanol, more preferably cyclohexanol and cyclododecanol.

Suitable catalysts are iron, cobalt, nickel, copper and vanadium, preferably cobalt, copper and vanadium, more preferably copper and vanadium.

Suitable nitric acid is aqueous nitric acid such as 20 to 98% by weight, preferably 30 to 80% by weight, more preferably 40 to 70% by weight and especially 55 to 65% by weight nitric acid.

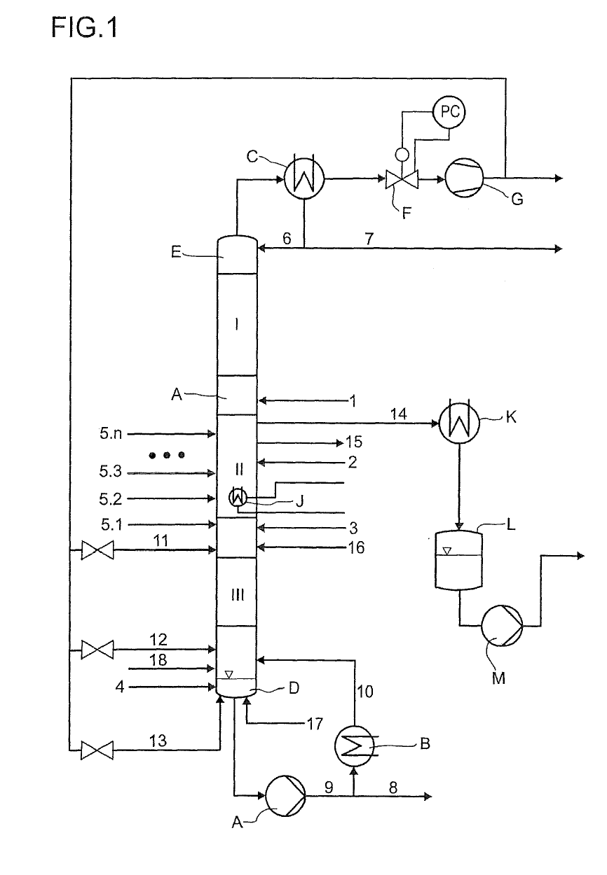

An advantageous version of the process according to the invention is described hereinafter with reference to FIG. 1. The apparatus, parameters and boundary conditions described therein also apply to the general description of the present invention.

The process according to the invention can appropriately be performed by metering in the aqueous nitric acid together with a catalyst via feed (1) and/or feed (2) onto the internals of section (II) of a fractionating column (A) which functions as a reaction column and rectification column. In addition, a portion of the nitric acid can be introduced via the feed (3) below section (II) of the column or via feed (4) into the bottom of the column. It may likewise be advantageous to feed recycle streams which may comprise product and by-products, nitric acid, water and catalyst in different composition to the column via feeds (1) to (4).

The reactant consisting generally of cycloalkanone(s), cycloalcohol(s) or mixtures thereof in different composition and can preferably be fed to the column separately from the nitric acid via feed streams (5.n) where n=1 to infinity, likewise onto the internals of section (II), or completely or partly directly below section (II). It may be advantageous to add cycloalkanone(s), cycloalcohol(s) or preferably mixtures thereof of entirely or partly in gaseous or liquid form via feed streams (5.n) where n=1 to infinity. The number of addition sites of cycloalkanone(s), cycloalcohol(s) or mixtures thereof in section (II) of the column is generally between 1 and infinity, i.e. from 1 to 100, preferably from 1 to 30, more preferably from 1 to 8 and especially 1 to 3 addition sites. The addition can be effected completely or partly into the liquid phase or gas phase in the region of the internals (II). It is advantageous to introduce cycloalkanone(s), cycloalcohol(s) or mixtures thereof into the column in very finely distributed form, for example by means of nozzles, tubes with holes with suitable apparatus such as nozzles, liquid distributors, nozzle tubes with holes. Suitable apparatus has been found to be ring or rod distributors with finely distributed bores of different diameter depending on the throughput of between 10 μm and 20 mm, preferably between 100 μm and 5 mm, more preferably between 200 mm and 1 mm. Likewise suitable are nozzles in known embodiments, for example one-substance, two-substance or multisubstance nozzles, hollow cones, full cones, flat-jet or glass-jet nozzles from Lechler, Schlick, Spraying Systems or other manufacturers, or any other kind of distributor constructions which ensure very fine and homogeneous distribution of liquid or gaseous reactant mixture, such as nozzle distributors, hole distributors with and without initial pressure, overflow distributors, perforated tray distributors, perforated channel distributors with and without predistribution, distributors with dripping fingers, channel groove distributors. It may be advantageous to combine the metering elements with static mixing elements.

The reaction of the cycloalkanone(s), cycloalcohol(s) or mixtures thereof with the nitric acid may take place on the internals of section (II) of the fractionating column. As a result of the distillation in the column, the reaction products formed are generally removed continuously from reaction section (II). The dicarboxylic acids formed generally pass together with a portion of the nitric acid as high-boiling components into the bottom (D) of the fractionating column, and can be drawn off via stream (8). Establishment of a suitable reflux ratio by adjusting the energy input through the evaporator (B) and condenser (C) and of the ratio between reflux (6) and distillate stream (7) allows the desired nitric acid concentration in the reaction section (II) of the column to be established through the distillative action of the column. Variation of the draw streams (7) and (8) additionally allows the concentration of the reaction product in the bottoms to be adjusted. The low-boiling components, water and a portion of the nitric acid, pass into the upper section (I) of the column through the distillative action.

It is advantageous but not obligatory when the nitric acid reactant, which boils at relatively high temperatures, is fed, separately or together with the catalyst, batchwise, preferably continuously, into the fractionating column (A) above the mixture of cycloalkanones and cycloalcohols which boils at lower temperatures by a suitable division, for example, into equal parts or different amounts, by means of control valves and flow meters or volume meters and the corresponding control devices, between feed streams (1) to (5), and a countercurrent flow of the reactants is established in this way. The pressure at the top of the column (E) is set at absolute pressure between 10 and 1000 mbar, preferably at absolute pressure between 100 and 200 mbar. According to the system, this can be done, for example, with a vacuum pump (G) and/or a pressure regulating device (F).

The column (A) consists generally of a plurality of zones with different functions. On the column internals of the above-described reaction zone (II), essentially the conversion of the reactants proceeds with simultaneous distillative removal of the products formed. Above the segment (II) is disposed the distillation zone (I), which is provided with distillative separating elements such as random packings, structured packings or trays. In this zone, nitric acid is separated from water. Owing to the thermodynamic boiling behavior, the nitric acid passes back into section (II) of column (A), while the water is obtained in gaseous form at the top of column (E), condensed in the condenser (C) and fed partly into column (A) via the recycle stream (6) and discharged partly via the draw stream (7). In this way, by virtue of the internal reflux in the column, an advantageous concentration profile can be established. A reflux ratio between 0:1 and 100:1, preferably between 0.1:1 and 5:1, should be established.

According to the number, type and size of internals such as random packings, structured packings or trays, and as a result of establishment of a suitable reflux ratio generally between 0.01 and 100 by adjusting the energy input through the evaporator (B) and condenser (C), the stream (7) drawn off comprises greater or lesser residual concentrations of nitric acid. Other high-boiling components such as catalyst or reaction products, which might be entrained from zone (II) into zone (I) in parts of column (A) owing to a high gas velocity, are likewise precipitated in zone (I) and are recycled back into the lower part of the column. Above it or else partly within it, a substream can be withdrawn from the column in gaseous or liquid form via the side draw streams (14) and (15). Gaseous draw removal is advantageous especially when very highly concentrated nitric acid is used or when the bottom product should comprise very high concentrations of product. In that case, nitric acid can be drawn off in very pure concentration via a gaseous draw. The gaseous stream (15) is condensed in a condenser (K), collected in vessel (L) and drawn off via pump (M). Instead of the gaseous side draw, the nitric acid can also be effected via the middle draw of a dividing wall column. The reaction then takes place on the feed section of the dividing wall column, while the nitric acid is concentrated and drawn off in the removal section.

Below segment (II) is disposed zone (III) which, like zone (I), is provided with distillative separating elements, for example random packings, structured packings or trays. In this zone, the reaction product is concentrated, such that the product concentration desired for the further workup is achieved in the bottom (D) of column (A). Product concentrations between 0 and 90% by weight, preferably between 10 and 50% by weight and more preferably between 20 and 40% by weight can be achieved. Excess nitric acid passes, as a result of the distillative action, back into zone (II) of column (A) and can react there again with the cycloalkanone and cycloalkane reactants supplied.

The high-boiling reaction product from the bottom (D) of the column (A) can be drawn off via the bottom stream (9) together with nitric acid by means of a pump (H). A portion of the bottom stream (9) can be evaporated with an evaporator (B) and conducted into the column via the vapor line (10). As a result, the vapors required for the distillation are generated.

The nitrogen oxides formed in the reaction in zone (II) go, as a volatile gas, momentarily into the gas phase via zone (I) of the column upward into the top (E) of the column (A). In the case of suitable selection of the heat carrier temperature (temperature at which the NO is not converted to the liquid phase at the selected pressure, but rather remains in gaseous form) in the condenser (C) of the column (A), the nitrogen oxide is not condensed out and passes together with other gases (especially nitrogen) to the vacuum pump (G). The offgas then comprises nitrogen oxides, for example nitrogen dioxide, oxygen and possibly nitrogen. By means of the vacuum pump (G), the offgas can then be recycled completely or partly into column (A). Suitable feed sites have been found to be the feed streams (11), (12) and (13). As a result of this recycling, the nitrous gas passes into zone (II) of the reactor. Here, as well as nitrous acid, nitric acid is formed continuously from the nitrogen dioxide of the recycled gas and in turn again oxidizes the particular cyclo compound. Addition of oxygen or oxygenous gases via feed streams (16), (17) and/or (18) allows the oxidation of nitrogen monoxide to nitrogen dioxide to be accelerated. The supply via feed streams (11) and (16), which are disposed between zone (II) and zone (III) of column (A), prevents the product formed from still comprising dissolved gas. According to the amount of gas recycled, zone (III) or the bottom (D) of column (A) has the effect that gases remain dissolved in the liquid product. According to the operating conditions, it is, however, also sufficient to introduce the recycled nitrous gases or the oxygenous gases via feed streams (18) and (12) into the gas phase of the bottom (D) of column (A), without there being any dissolution of gas in the liquid in the bottom (D) of the column (A). An advantage of the addition via this site is that nitric acid and/or NO₂ can form from NO as early as in zone (III). In this case, in zone (III), as well as the distillation, a reaction likewise takes place. The yield of nitric acid can thus be enhanced. In addition, it is possible to introduce gas (air or recycled nitrous gases) into the column via streams (13) or (17). In this case, however, a downstream degassing step is needed. An improvement in the mixing is not achieved, since the bottoms are mixed efficiently from the outset by the forced circulation of the pump (H) via the evaporator (B). The circulation can also be effected without pump (H) with a natural circulation. Alternatively, a heated stirred tank can of course also be used.

In the reaction zone (II), the internals used are advantageously distillation trays such as sieve trays or trays with a high residence time of the liquid, for example valve trays, preferably bubble-cap trays or related designs, for example tunnel-cap trays or Thormann trays. Alternatively, it is possible to use metal fabric packings or sheet metal packings with ordered structure, or else random packings, as column internals. The above-described devices for metering in the cycloalkanones or-alcohols are combined with these internals. This can be done between the internals or directly on the internals in the gas or the liquid phase. It is advantageous, for example, to use reaction trays with a long residence time and defined flow direction (e.g. Thormann trays), onto which are combined with metering lances and/or cooling or heating coils (JI). The heat carrier coils can be used for additional removal of heat of reaction, or for startup and shutdown operations. In the case of sufficient liquid hold on the internals in the region of the reaction, this is, however, generally not necessary. The liquid holdup should always be greater than the amount of liquid by which heat of reaction released in the reaction can evaporate within a defined period (e.g. residence time).

It is advantageous to meter in the reactant mixture in very finely distributed form and homogeneously through the column tray. This can be done, for example, by virtue of the metering lances being manufactured with holes with different bore diameters. It may be advantageous for the mixing to provide the metering lances with static mixing elements.

To perform the reaction, advantageously, fractionating columns which, with their internals, have from 1 to 150 theoretical plates, preferably from 2 to 100 theoretical plates, more preferably from 3 to 50 theoretical plates, especially from 4 to 20 theoretical plates, are used. It has been found to be particularly advantageous when from 1 to 5 theoretical plates, preferably from 2 to 4 theoretical plates, are provided in reaction zone (III), from 1 to 5 theoretical plates, preferably from 2 to 4 theoretical plates, in zone (II), and from 1 to 10 theoretical plates, preferably from 2 to 7 theoretical plates, in the upper part of the column (I) above the feed stream (1).

The process according to the invention can in principle be applied to all chemical syntheses for the preparation of dicarboxylic acids proceeding from cycloalkanones and/or cycloalcohols with aqueous acid. However, the process is of particular significance for the oxidation of cyclohexanone and cyclohexanol to adipic acid.

The process according to the invention can afford the products, in the case of complete conversion of the cycloalkanones and/or cycloalkanes, in high selectivity with simultaneous removal of excess nitrous gases, water and/or nitric acid.

### EXAMPLES

### Example 1

### Oxidation of a Mixture of Cyclohexanol and Cyclohexanone

### Description of the Apparatus

The test apparatus consisted of a heatable 2 liter reaction flask made of stainless steel, to which was attached a distillation column (length: 1.5 m, diameter: 50 mm). The column was filled with 3 bubble-cap trays in the lower section (zone (III)), with 5 bubble-cap trays in the middle section (zone (II)) and with 6 bubble-cap trays in the upper section (zone (I)). The reaction solution (mixture of cyclohexanol and cyclohexanone) was fed in via an inserted tube provided with several holes to the 4th bubble-cap tray (counted from the bottom). The column was equipped with three thermocouples, such that, except in the bottom (in the liquid) and at the top of the column (gas section before the condenser), the temperature in the liquid phase could be measured in reaction section (II).

The reactants were metered into the column under mass flow control from reservoir vessels resting on balances with a pump. The evaporator (B) was heated with the aid of a thermostat. The bottom stream (8) was conveyed out of the evaporator with a pump under level control into a vessel resting on a balance. The top stream of the column was condensed in a condenser (C) at temperatures between 40 and 60° C. A portion (7) of the condensate was delivered with a pump into a distillate vessel resting on a balance, while the other portion (6) was introduced as reflux to the column. The apparatus was equipped with a pressure regulator (F1) which was able to regulate a pressure of from 50 to 300 mbar. The temperature in the section region (II) of the column was regulated to a given temperature by adjusting the heating output and the reflux rate. It was thus possible to adjust the nitric acid concentration in the reaction section. All entering and exiting streams were detected continuously with a PCS and registered over the entire test. The apparatus was operated for 8 hours (steady-state operation). After approx. 4 hours, a steady state was established.

### Test Procedure

1000 g/h of an aqueous nitric acid solution (37%) were introduced continuously to the 8th tray (from the bottom) of the column. A copper/vanadium catalyst was dissolved in this solution. 22 g/h of a mixture of 15% by weight of cyclohexanone and 85% by weight of cyclohexanol were pumped continuously to the 4th tray. A system pressure of 195 mbar and a reflux rate of approx. 429 g/h were established. The temperature in the reaction section (II) of the column was regulated at 75° C. by adjusting the heating output and the reflux rate. The HNO₃ content thus achieved on the reaction tray was approx. 47% by weight. The bottom temperature was 82° C. The gas temperature upstream of the condenser was 59° C. The bottom stream obtained from the column was 551 g/h of crude product with 59.6% by weight of HNO₃, 5.2% by weight of adipic acid (ADA), 0.5% by weight of glutaric acid, traces (<0.1% by weight) of succinic acid and water. At the top of the column, 449 g/h of distillate consisting of water were drawn off. The nitrous gases were led off in gaseous form via a flare. ADA was obtained with a selectivity of 90% based on the mixture of cyclohexanone and cyclohexanol, and glutaric acid with a selectivity of 10% based on the mixture of cyclohexanone and cyclohexanol. The mixture of cyclohexanone and cyclohexanone was converted fully.

### Example 2

### Oxidation of a Mixture of Cyclohexanol and Cyclohexanone

In the apparatus described in Example 1, 1021 g/h of an aqueous nitric acid solution (37%) were introduced continuously to the 8th tray (from the bottom) of the column. A copper/vanadium catalyst was dissolved in this solution. 25 g/h of a mixture of 15% by weight of cyclohexanone and 85% by weight of cyclohexanol were pumped continuously to the 4th tray. A system pressure of 153 mbar and a reflux rate of approx. 667 g/h were established. The temperature in the reaction section (II) of the column was regulated at 74° C. by adjusting the heating output and the reflux rate. The HNO₃ content thus achieved on the reaction tray was approx. 56% by weight. The bottom temperature was 77° C. The gas temperature upstream of the condenser was 50° C. The bottom stream obtained from the column was 560 g/h of crude product with 59.0% by weight of HNO₃, 6.2% by weight of adipic acid (ADA), 0.4% by weight of glutaric acid, 0.03% by weight of succinic acid and water. At the top of the column, 461 g/h of distillate consisting of water were drawn off. The nitrous gases were led off in gaseous form via a flare. ADA was obtained with a selectivity of 93.3% based on the mixture of cyclohexanone and cyclohexanol, glutaric acid with a selectivity of 6.2% based on the mixture of cyclohexanone and cyclohexanol, and succinic acid with a selectivity of 0.5% based on the mixture of cyclohexanone and cyclohexanol. The mixture of cyclohexanone and cyclohexanol was converted fully.

### Example 3

### Oxidation of a Mixture of Cyclohexanol and Cyclohexanone

In the apparatus described in Example 1, 1000 g/h of an aqueous nitric acid solution (37%) were introduced continuously to the 8th tray (from the bottom) of the column. A copper/vanadium catalyst was dissolved in this solution. 23 g/h of a mixture of 40% by weight of cyclohexanone and 60% by weight of cyclohexanol were pumped continuously to the 4th tray. A system pressure of 126 mbar and a reflux rate of approx. 429 g/h were established. The temperature in the reaction section (II) of the column was regulated at 71° C. by adjusting the heating output and the reflux rate. The HNO₃ content thus achieved on the reaction tray was approx. 60% by weight. The bottom temperature was 74° C. The gas temperature upstream of the condenser was 47° C. The bottom stream obtained from the column was 553 g/h of crude product with 59.0% by weight of HNO₃, 5.8% by weight of adipic acid (ADA), 0.34% by weight of glutaric acid, 0.03% by weight of succinic acid and water. At the top of the column, 447 g/h of distillate consisting of water were drawn off. The nitrous gases were led off in gaseous form via a flare. ADA was obtained with a selectivity of 93.3% based on the mixture of cyclohexanone and cyclohexanol, glutaric acid with a selectivity of 6.1% based on the mixture of cyclohexanone and cyclohexanol, and succinic acid with a selectivity of 0.6% based on the mixture of cyclohexanone and cyclohexanol. The mixture of cyclohexanone and cyclohexanol was converted fully.

### Example 4

### Oxidation of a Mixture of Cyclohexanol and Cyclohexanone

In the apparatus described in Example 1, 1003 g/h of an aqueous nitric acid solution (37%) were introduced continuously to the 8th tray (from the bottom) of the column. A copper/vanadium catalyst was dissolved in this solution. 23 g/h of a mixture of 15% by weight of cyclohexanone and 85% by weight of cyclohexanol were pumped continuously to the 4th tray. A system pressure of 125 mbar and a reflux rate of approx. 2114 g/h were established. The temperature in the reaction section (II) of the column was regulated at 71° C. by adjusting the heating output and the reflux rate. The HNO₃ content thus achieved on the reaction tray was approx. 60% by weight. The bottom temperature was 74° C. The gas temperature upstream of the condenser was 47° C. The bottom stream obtained from the column was 557 g/h of crude product with 59.0% by weight of HNO₃, 5.9% by weight of adipic acid (ADA), 0.15% by weight of glutaric acid, 0.03% by weight of succinic acid and water. At the top of the column, 448 g/h of distillate consisting of water were drawn off. The nitrous gases were led off in gaseous form via a flare. ADA was obtained with a selectivity of 96.6% based on the mixture of cyclohexanone and cyclohexanol, glutaric acid with a selectivity of 2.8% based on the mixture of cyclohexanone and cyclohexanol, and succinic acid with a selectivity of 0.6% based on the mixture of cyclohexanone and cyclohexanol. The mixture of cyclohexanone and cyclohexanol was converted fully.

### Example 5

### Oxidation of a Mixture of Cyclohexanol and Cyclohexanone

The apparatus described in Example 1 was equipped with additional heating elements in the bottom section of the column. In addition, all pipelines and fittings in the lower part of the column were trace-heated in order to prevent precipitation of solid.

942 g/h of an aqueous nitric acid solution (37%) were introduced continuously to the 8th tray (from the bottom) of the column. A copper/vanadium catalyst was dissolved in this solution. 38 g/h of a mixture of 15% by weight of cyclohexanone and 85% by weight of cyclohexanol were pumped continuously to the 4th tray. A system pressure of 125 mbar and a reflux rate of approx. 482 g/h were established. The temperature in the reaction section (II) of the column was regulated at 71° C. by adjusting the heating output and the reflux rate. The HNO₃ content thus achieved on the reaction tray was approx. 60% by weight. The bottom temperature was 74° C. The gas temperature upstream of the condenser was 47° C. The bottom stream obtained from the column was 497 g/h of crude product with 56.0% by weight of HNO₃, 10.7% by weight of adipic acid (ADA), 0.32% by weight of glutaric acid, 0.07% by weight of succinic acid and water. At the top of the column, 446 g/h of distillate consisting of water were drawn off. The nitrous gases were led off in gaseous form via a flare. ADA was obtained with a selectivity of 95.8% based on the mixture of cyclohexanone and cyclohexanol, glutaric acid with a selectivity of 3.5% based on the mixture of cyclohexanone and cyclohexanol, and succinic acid with a selectivity of 0.7% based on the mixture of cyclohexanone and cyclohexanol. The mixture of cyclohexanone and cyclohexanol was converted fully.

### Example 6

### Oxidation of a Mixture of Cyclohexanol and Cyclohexanone

The apparatus described in Example 1 was equipped with additional heating elements in the bottom section of the column. In addition, all pipelines and fittings in the lower part of the column were trace-heated in order to prevent precipitation of solid as a result of cooling and crystallization.

942 g/h of an aqueous nitric acid solution (37%) were introduced continuously to the 8th tray (from the bottom) of the column. A copper/vanadium catalyst was dissolved in this solution. 89 g/h of a mixture of 15% by weight of cyclohexanone and 85% by weight of cyclohexanol were pumped continuously to the 4th tray. A system pressure of 125 mbar and a reflux rate of approx. 495 g/h were established. The temperature in the reaction section (II) of the column was regulated at 71° C. by adjusting the heating output and the reflux rate. The HNO₃ content thus achieved on the reaction tray was approx. 57% by weight. The bottom temperature was 75° C. The gas temperature upstream of the condenser was 47° C. The bottom stream obtained from the column was 420 g/h of crude product with 43.5% by weight of HNO₃, 29.8% by weight of adipic acid (ADA), 1.6% by weight of glutaric acid, 0.2% by weight of succinic acid and water. At the top of the column, 520 g/h of distillate consisting of water were drawn off. The nitrous gases were led off in gaseous form via a flare. ADA was obtained with a selectivity of 93.7% based on the mixture of cyclohexanone and cyclohexanol, glutaric acid with a selectivity of 5.6% based on the mixture of cyclohexanone and cyclohexanol, and succinic acid with a selectivity of 0.8% based on the mixture of cyclohexanone and cyclohexanol. The mixture of cyclohexanone and cyclohexanol was converted fully.

### Example 7

### Oxidation of a Mixture of Cyclohexanol and Cyclohexanone

The apparatus described in Example 1 was equipped with additional heating elements in the bottom section of the column. In addition, all pipelines and fittings in the lower part of the column were trace-heated in order to prevent precipitation of solid as a result of cooling and crystallization.

987 g/h of an aqueous nitric acid solution (37%) were introduced continuously to the 8th tray (from the bottom) of the column. A copper/vanadium catalyst was dissolved in this solution. 97 g/h of a mixture of 15% by weight of cyclohexanone and 85% by weight of cyclohexanol were pumped continuously to the 4th tray. A system pressure of 125 mbar and a reflux rate of approx. 2185 g/h were established. The temperature in the reaction section (II) of the column was regulated at 72° C. by adjusting the heating output and the reflux rate. The HNO₃ content thus achieved on the reaction tray was approx. 61% by weight. The bottom temperature was 75° C. The gas temperature upstream of the condenser was 47° C. The bottom stream obtained from the column was 435 g/h of crude product with 43.0% by weight of HNO₃, 31.6% by weight of adipic acid (ADA), 0.8% by weight of glutaric acid, 0.2% by weight of succinic acid and water. At the top of the column, 551 g/h of distillate consisting of water were drawn off. The nitrous gases were led off in gaseous form via a flare. ADA was obtained with a selectivity of 96.5% based on the mixture of cyclohexanone and cyclohexanol, glutaric acid with a selectivity of 2.7% based on the mixture of cyclohexanone and cyclohexanol, and succinic acid with a selectivity of 0.8% based on the mixture of cyclohexanone and cyclohexanol. The mixture of cyclohexanone and cyclohexanol was converted fully.

1. A process for preparing dicarboxylic acids by oxidizing cycloaliphatic alcohols, cycloaliphatic ketones or mixtures thereof with nitric acid, which comprises performing the reaction and separation of the components in a fractionating column, reaction column and/or rectification column.

2. The process for preparing dicarboxylic acids according to claim 1, wherein the fractionating column, reaction column and/or rectification column comprises from 1 to 150 theoretical plates.

3. The process for preparing dicarboxylic acids according to claim 2, wherein the fractionating column, reaction column and/or rectification column comprises from 3 to 20 segments with internals.

4. The process for preparing dicarboxylic acids according to claim 3, wherein the segments with internals in the fractionating column, reaction column and/or rectification column are separated by intermediate spaces.

5. The process for preparing dicarboxylic acids according to claim 4, wherein the length ratio of intermediate space relative to the segment below it in the fractionating column, reaction column and/or rectification column is from 0.01:1 to 10:1.

6. The process for preparing dicarboxylic acids according to claim 1, wherein the fractionating column, reaction column and/or rectification column comprises from 3 to 20 segments with internals.

7. The process for preparing dicarboxylic acids according to claim 1, wherein the segments with internals in the fractionating column, reaction column and/or rectification column are separated by intermediate spaces.

8. The process for preparing dicarboxylic acids according to claim 1, wherein the length ratio of intermediate space relative to the segment below it in the fractionating column, reaction column and/or rectification column is from 0.01:1 to 10:1.
